SEQUENCE LISTING (4 sequences)

SEQ ID NO: 1 - RNA, 21 nt, synthetic construct
  source 1..21: mol_type = other RNA; note = sense strand of p55gamma-si1
gaaacagtccttcattccgtt

SEQ ID NO: 2 - RNA, 21 nt, synthetic construct
  source 1..21: mol_type = other RNA; note = antisense strand of p55gamma-si1
cggaatgaaggactgtttcgt

SEQ ID NO: 3 - RNA, 25 nt, synthetic construct
  source 1..25: mol_type = other RNA; note = sense strand of p55gamma-si2
gagacatttccagggaagaggtaaa

SEQ ID NO: 4 - RNA, 25 nt, synthetic construct
  source 1..25: mol_type = other RNA; note = antisense strand of p55gamma-si2
tttacctcttccctggaaatgtctc

(19) United States
(12) Patent Application Publication
(10) Pub. No.: US 2025/0283058 A1
(43) Pub. Date: Sep. 11, 2025

(54) USE OF p55-GAMMA GENE AND/OR p55-GAMMA PROTEIN AS TARGET IN MAINTAINING CARDIAC IRON HOMEOSTASIS AND TREATING RELATED DISEASE

(71) Applicant: BEIJING FRIENDSHIP HOSPITAL, CAPITAL MEDICAL UNIVERSITY, Beijing (CN)

(72) Inventors: Chunmei CAO, Beijing (CN); Kun ZHU, Beijing (CN); Yangli LIU, Beijing (CN); Rilei DAI, Beijing (CN)

(73) Assignee: BEIJING FRIENDSHIP HOSPITAL, CAPITAL MEDICAL UNIVERSITY, Beijing (CN)

(21) Appl. No.: 18/741,178

(22) Filed: Jun. 12, 2024

(30) Foreign Application Priority Data
Mar. 5, 2024 (CN) 202410251028.6

(51) Int. Cl. C12N 9/12; A61K 31/445; A61K 38/00; A61K 48/00; A61P 9/00; C12N 15/86; G01N 33/50

(52) U.S. Cl. CPC C12N 9/1205; A61K 31/445; A61K 48/0008; A61P 9/00; C12N 15/86; G01N 33/5023; A61K 38/00; C12N 2710/10043; C12Y 207/01137

(57) ABSTRACT

Provided is use of p55γ gene and/or p55γ protein as a target in maintaining cardiac iron homeostasis and treating a related disease, which belongs to the technical field of biomedicine. In the present disclosure, a p55γ transgenic mice model and an ischemia/reperfusion (I/R) injury model in vivo and in vitro are used, and it is proposed that overexpression of p55γ alleviates I/R injury induced cardiac ferroptosis, thereby protecting cardiac function. Moreover, it is found that the overexpression of p55γ leads to downregulation of transferrin receptor 1 (Tfr1) by downregulating iron-regulatory protein 2 (IRP2), thereby maintaining the cardiac iron homeostasis to resist myocardial I/R injuries. This demonstrates a potential of the p55γ gene and/or the p55γ protein as a key regulator of I/R injury induced cardiomyocyte death, thereby providing new targets for maintaining the cardiac iron homeostasis and developing novel therapeutic approaches for related diseases.

### CROSS REFERENCE TO RELATED APPLICATION(S)

This patent application claims the benefit and priority of Chinese Patent Application No. 202410251028.6 filed with the China National Intellectual Property Administration on Mar. 5, 2024, the disclosure of which is incorporated by reference herein in its entirety as part of the present application.

### REFERENCE TO SEQUENCE LISTING

A computer readable XML file entitled “HLP20240100446_seqlist”, that was created on May 21, 2024, with a file size of about 5,254 bytes, contains the sequence listing for this application, has been filed with this application, and is hereby incorporated by reference in its entirety.

### TECHNICAL FIELD

The present disclosure belongs to the technical field of biomedicine, and specifically relates to use of p55γ gene and/or p55γ protein as a target in maintaining cardiac iron homeostasis and treating a related disease.

### BACKGROUND

The maintenance of iron homeostasis is crucial for normal cardiac functions. Ferroptosis is a form of programmed cell death caused by iron-dependent phospholipid peroxidation. Studies have shown that iron metabolism, glutathione metabolism, and lipid metabolism are key players in ferroptosis. Iron metabolism involves the absorption, excretion, storage, and utilization of iron. Iron-regulatory proteins (IRPs) induce the upregulation of transferrin receptor (TfR) expression and the downregulation of ferritin and ferroportin (FPN) expression by binding to iron-responsive elements (IREs), thereby maintaining cellular iron homeostasis. Imbalances in iron homeostasis are associated with a variety of cardiovascular diseases, including atherosclerosis, myocardial ischemia/reperfusion (I/R) injury, arrhythmias, and diabetic cardiomyopathy. Although excess iron accumulation, disruption of iron homeostasis, and abnormal expression of iron homeostasis-related proteins are observed in the heart during myocardial injury, the mechanisms underlying iron metabolism and regulation remain unclear. Therefore, a deeper understanding of the regulation of iron metabolism and ferroptosis in the heart is of great significance for the treatment of these diseases.

Ischemic heart disease is the leading cause of cardiovascular death. Early and timely revascularization is a common treatment for the ischemic heart disease, but may cause additional myocardial damage-I/R injury, resulting in death in a significant proportion of patients. The molecular mechanisms of I/R injury involve coronary microvascular damage and cardiomyocyte death. Despite extensive research on the molecular mechanisms of I/R injury, there are currently no approved pharmacological interventions to treat I/R injury. Programmed cell death, including necrosis, apoptosis, autophagy, necroptosis, pyroptosis, and ferroptosis, has been identified as an important mechanism in the occurrence and progression of myocardial I/R injury. However, the molecular mechanisms regulating various forms of cell death during I/R injury are not fully understood.

p55γ, as a regulatory subunit of phosphatidylinositol 3-kinase (PI3K), is encoded by a Pik3r3 gene. Previous studies have shown that p55γ affects the occurrence and development of tumors by regulating cell proliferation, cell cycle, and cell survival. Contrary to its function in tumors, p55γ inhibits vascular smooth muscle cell proliferation, thereby inhibiting balloon injury-induced arterial neointima formation. These findings suggest that the pathophysiological functions of p55γ are cell type specific, but there are no reports on its role in I/R-induced myocardial ferroptosis as well as its involvement in maintaining cardiac iron homeostasis and related diseases.

### SUMMARY

The present disclosure provides use of p55γ gene and/or p55γ protein as a target in maintaining cardiac iron homeostasis and treating a related disease. Overexpression of the p55γ gene may effectively maintain the cardiac iron homeostasis and resist myocardial I/R injuries. Targeting the p55γ gene holds promise as a new strategy for the treatment of cardiovascular diseases related to iron homeostasis imbalance.

The present disclosure provides the following technical solutions:

The present disclosure provides use of p55γ gene and/or p55γ protein as a target in preparation of a drug for maintaining cardiac iron homeostasis and treating a related disease.

In some embodiments, promotion of activity of the p55γ protein or promotion of expression of the p55γ gene maintains the cardiac iron homeostasis and treat the related disease.

In some embodiments, transfection of an adenovirus overexpressing the p55γ gene maintains the cardiac iron homeostasis and treats the related disease.

In some embodiments, overexpression of the p55γ gene leads to downregulation of transferrin receptor 1 (Tfr1) through an iron-regulatory protein 2 (IRP2) post-transcriptional system, thereby maintaining the cardiac iron homeostasis and treating the related disease.

In some embodiments, the p55γ protein increases ubiquitination and subsequent degradation of IRP2 by binding to a tripartite motif containing 28 (TRIM28) protein, thereby helping to downregulate expression of the Tfr1 and ultimately inhibiting ferroptosis to maintain the cardiac iron homeostasis and treat the related disease.

The present disclosure further provides use of p55γ protein in preparation of a drug for maintaining cardiac iron homeostasis and treating a related disease, where the p55γ protein inhibits cardiomyocyte ferroptosis.

The present disclosure further provides use of p55γ gene and/or p55γ protein as a target in screening a drug for maintaining cardiac iron homeostasis and treating a related disease, where the drug promotes expression of the p55γ gene and/or promotes activity of the p55γ protein to maintain the cardiac iron homeostasis and treat the related disease.

In some embodiments, the drug includes perhexiline.

Compared with the prior art, the embodiment of present disclosure has the following beneficial effects:

(1) In the present disclosure, it is proposed for the first time that overexpression of p55γ gene can alleviate cardiac ferroptosis induced by I/R injuries and protect cardiac functions. Moreover, it is found that the overexpression of the p55γ gene leads to downregulation of transferrin receptor 1 (Tfr1) by downregulating iron-regulatory protein 2 (IRP2), thereby maintaining the cardiac iron homeostasis to resist myocardial I/R injuries. The p55γ gene increases ubiquitination and subsequent degradation of IRP2 by binding to the TRIM28 protein, thereby helping to downregulate the expression of Tfr1 and ultimately inhibiting ferroptosis. These results indicate that the p55γ gene is important for maintaining cardiac iron homeostasis and is a novel inhibiting factor of cardiomyocyte ferroptosis during the I/R injury. Targeting the p55γ gene holds promise as a new strategy for the treatment of cardiovascular diseases related to iron homeostasis imbalance.

(2) In the present disclosure, it is also proposed that the p55γ protein is a novel inhibiting factor of cardiomyocyte ferroptosis. Administration of the p55γ protein may inhibit cardiomyocyte ferroptosis, thereby maintaining the cardiac iron homeostasis and treating the related disease.

### BRIEF DESCRIPTION OF THE DRAWINGS

FIGS. 1A-1F show that the p55γ gene expression is down-regulated in myocardial ferroptosis induced by ferroptosis inducers and inhibits erastin-induced myocardial ferroptosis; where (FIG. 1A) shows the mRNA level of the p55γ gene in cardiomyocytes after erastin treatment (n=11); (FIG. 1B) shows representative Western blot and average data of the p55γ gene in cardiomyocytes after erastin treatment (n=10); (FIG. 1C) shows the mRNA level of the p55γ gene in cardiomyocytes after RSL3 treatment (n=10); (FIG. 1D) shows representative Western blot and average data of the p55γ gene in cardiomyocytes after RSL3 treatment (n=8); (FIG. 1E) is a representation of the Western blot data of the p55γ gene in cardiomyocytes infected with Ad-p55γ for 48 h; (FIG. 1F) shows the cell viability (n=12), ptgs2 mRNA level (n=12), and intracellular MDA content (n=13) of cardiomyocytes infected with Ad-p55γ after erastin stimulating; and in (FIGS. 1A-1D and 1F), the data are expressed as the mean ±standard deviation of 3 to 4 independent experiments;

FIGS. 2A-2D show that p55γ protein inhibits cardiac ferroptosis induced by I/R injury; where (FIG. 2A) shows the cardiac infarct size (IF) and area at risk (AAR) of wild-type (WT) mice, Fer1 and DXZ-treated mice, and p55γ^{h-TG} mice after I/R injury (n=10); (FIG. 2B) shows the serum LDH concentration of WT mice, Fer1 and DXZ-treated mice, and p55γ^{h-TG} mice after I/R injury (n=10); (FIG. 2C) shows cardiac MDA content of WT mice, Fer1 and DXZ-treated mice, and p55γ^{h-TG} mice after I/R injury (n=10); (FIG. 2D) shows the content of oxidized lipids in the hearts of WT mice and p55γ^{h-TG} mice after I/R injury (n=5); in (FIGS. 2A-2C), the data are expressed as the mean ±standard deviation of 3 to 4 independent experiments;

FIGS. 3A-31 show that the p55γ gene inhibits cardiac ferroptosis induced by I/R injury by downregulating the expression of Tfr1; (FIG. 3A) shows the mRNA level of Tfr1 in the hearts of WT and p55γ^{h-TG} mice (n=10); (FIG. 3B) is a representation of the Western blot and average data for Tfr1 in the hearts of WT and p55γ^{h-TG} mice (n=5); (FIG. 3C) shows the mRNA level of Tfr1 in cardiomyocytes 48 h after Ad-p55γ infection of cardiomyocytes (n=8); (FIG. 3D) is a representation of the Western blot and average data of Tfr1 in cardiomyocytes 48 h after Ad-p55γ infection of cardiomyocytes (n=10); (FIG. 3E) shows the mRNA level of Tfr1 in cardiomyocytes 72 h after p55γ siRNA infection of cardiomyocytes (n=8); (FIG. 3F) is a representation of the Western blot and average data of Tfr1 in cardiomyocytes 72 h after p55γ siRNA infection of cardiomyocytes (n=10); (FIG. 3G) is a representation of Western blot and average data of Tfr1 in hearts after AAV9-Tfr1 infected 4 weeks (n=8); (FIG. 3H) shows the cardiac infarct size (IF) and area at risk (AAR) of WT mice and p55γ^{h-TG} mice infected or not infected with AAV9-Tfr1 after I/R injury (n=10); (FIG. 3I) shows the ptgs2 mRNA level, MDA content, and iron content in the hearts of WT mice and p55γ^{h-TG} mice infected or not infected with AAV9-Tfr1 after I/R injury (n=9 in ptgs2 mRNA detection, n=9 in MDA content detection, and n=6 in iron content detection); and in (FIGS. 3A-3I), the data are expressed as the mean ±standard deviation of 3 to 4 independent experiments;

FIGS. 4A-4J show that overexpression of the p55γ gene downregulates Tfr1 through the IRP2 post-transcriptional system; where (FIG. 4A) shows the mRNA stability of Tfr1; (FIG. 4B) shows the mRNA level of IRP2 in cardiomyocytes 48 h after Ad-p55γ infection of cardiomyocytes (n=12); (FIG. 4C) is a representation of the Western blot and average data of IRP2 in cardiomyocytes 48 h after Ad-p55γ infection of cardiomyocytes (n=8); (FIG. 4D) shows the mRNA level of IRP2 in cardiomyocytes 72 h after p55γ siRNA infection of cardiomyocytes (n=10); (FIG. 4E) is a representation of the Western blot and average data of IRP2 in cardiomyocytes 72 h after p55γ siRNA infection of cardiomyocytes (n=8); (FIG. 4F) is a representation of the Western blot and average data of IRP2 in Ad-p55γ-infected cardiomyocytes treated with MG132 (n=8); (FIG. 4G) is a representation of the Western blot data of IRP2 ubiquitination after plasmid transfection in HEK293T cells (n=3); (FIG. 4H) is a representation of the Western blot data of IRP2 after Ad-IRP2 infection of cardiomyocytes; (FIG. 4I) is a representation of the Western blot and average data of Tfr1 in cardiomyocytes 48 h after Ad-p55γ and/or Ad-IRP2 infection of cardiomyocytes (n=8); (FIG. 4J) shows the ptgs2 mRNA level and cell viability of cardiomyocytes infected with Ad-p55γ and/or Ad-IRP2 after (hypoxia/reoxygenation) H/R injury (n=12); and in (FIGS. 4A-4F, 4I, and 4J), the data are expressed as the mean ±standard deviation of 3 to 4 independent experiments;

FIGS. 5A-5F show that p55γ inhibits cardiac ferroptosis by promoting TRIM28-mediated ubiquitination and degradation of IRP2; (FIG. 5A) shows the intersection of proteins identified by transfecting HA-tagged IRP2 into cardiomyocytes, and purifying complex containing IRP2 with HA antibody, followed by mass spectrometry analysis, and those identified as ubiquitin ligase E3 by UbiBrowser 2.0; and the table showing the mass spectrum information of the intersected proteins; (FIG. 5B) shows the co-immunoprecipitation of IRP2 and TRIM28 in cardiomyocytes (n=3); (FIG. 5C) shows the co-immunoprecipitation of p55γ and TRIM28 in cardiomyocytes (n=3); (FIG. 5D) shows the immunoprecipitation of purified TRIM28 protein, purified IRP2 protein, and p55γ protein (n=3); (FIG. 5E) is a representation of the Western blot data of IRP2 ubiquitination in HEK293T cells (n=3); (FIG. 5F) is a representation of the Western blot and average data of IRP2 and Tfr1 transfected with Ad-p55γ treated with TRIM28 siRNA (n=8 in IRP2 detection; n=6 in Tfr1 detection); and in (FIG. 5F), the data are expressed as the mean ±standard deviation of 3 to 4 independent experiments;

FIGS. 6A-6C show that TRIM28 promotes the polyubiquitination of the K48 chain of IRP2 at the K877 site; (FIG. 6A) is representative Western blot data of IRP2 ubiquitination in HEK293T cells (n=3); (FIG. 6B) is a representation of the Western blot data of IRP2 ubiquitination in HEK293T cells (n=3); and (FIG. 6C) is a representation of the Western blot data of IRP2 ubiquitination in HEK293T cells (n=3);

FIGS. 7A-7E show that p55γ activator Perhexiline inhibits cardiac ferroptosis induced by I/R injury; where (FIG. 7A) is a molecular docking model diagram of p55γ and Perhexiline; (FIG. 7B) is a representation of the Western blot and average data of Tfr1 and IRP2 after Perhexiline treatment of cardiomyocytes for 24 h (IRP2, n=6; Tfr1, n=7); (FIG. 7C) shows the cell activity of cardiomyocytes (n=12) and the ptgs2 mRNA level of cardiomyocytes (n=5) after constructing the H/R injury model under IRP2 adenovirus transfection of NRVMs with or without Perhexiline treatment; (FIG. 7D) is a representative picture showing 2,3,5-triphenyltetrazolium chloride (TTC) staining in mice injected intraperitoneally with Perhexiline after constructing an I/R injury model (n=10), and average data thereof; (FIG. 7E) shows the MDA content in the heart of mice injected intraperitoneally with Perhexiline after constructing an I/R injury model (n=5); and in (FIGS. 7B-7E), the data are expressed as the mean ±standard deviation of 3 to 4 independent experiments.

### DETAILED DESCRIPTION OF THE EMBODIMENTS

The present disclosure provides use of p55γ gene and/or p55γ protein as a target in preparation of a drug for maintaining cardiac iron homeostasis and treating a related disease. In the present disclosure, a p55γ transgenic mice model and an ischemia/reperfusion (I/R) injury model in vivo and in vitro are used, and it is proposed that overexpression of the p55γ gene may alleviate I/R injury-induced cardiac ferroptosis, thereby protecting cardiac functions. Moreover, the overexpression of the p55γ gene leads to downregulation of transferrin receptor 1 (Tfr1) by downregulating iron-regulatory protein 2 (IRP2), thereby maintaining the cardiac iron homeostasis to resist myocardial I/R injuries. The present disclosure further proposes that TRIM28 is a novel E3 ligase of IRP2. The p55γ protein increases ubiquitination and subsequent degradation of an IRP2 by binding to a TRIM28 protein, thereby helping to downregulate an expression of the Tfr1 and ultimately inhibiting ferroptosis to maintain the cardiac iron homeostasis and treat the related disease.

In the present disclosure, transfection of an adenovirus overexpressing the p55γ gene maintains the cardiac iron homeostasis and treats the related disease. The adenovirus overexpressing the p55γ gene is prepared based on an NM_003629.4 sequence in NCBI to the EcoRI/XhoI site of a pcDNA3.1(+) vector.

The present disclosure further provides use of the p55γ protein in preparation of a drug for maintaining cardiac iron homeostasis and treating a related disease, where the p55γ protein inhibits ferroptosis of a cardiomyocyte. In the present disclosure, the p55γ protein can be obtained through standard commercial sources or prepared by conventional preparation methods. The p55γ protein inhibits cardiomyocyte ferroptosis, thereby maintaining the cardiac iron homeostasis and treating the related disease. The disease according to the present disclosure includes cardiovascular diseases associated with iron homeostasis imbalance.

The present disclosure further provides use of the p55γ gene and/or the p55γ protein as a target in preparation of a drug for maintaining cardiac iron homeostasis and treating a related disease, where the drug promotes expression of the p55γ gene and/or promotes activity of the p55γ protein to maintain the cardiac iron homeostasis and treat the related disease. The drug includes perhexiline.

The present disclosure further provides a strategy for preparing a drug to maintain the cardiac iron homeostasis and treat the related disease. The drug includes one of the following: (1) an antibody against the p55γ protein; (2) a pharmaceutical preparation containing an agonist for the p55γ protein, or pharmaceutically acceptable salt, solvate, or hydrate thereof; (3) DNA or RNA that promotes the expression of p55γ protein. The drug is further preferably perhexiline. The drug further includes a pharmaceutically acceptable carrier of the above compound. The drug has a dosage form including injections, powder injections, pills, powder, tablets, patch preparations, suppositories, emulsions, creams, gels, granules, capsules, aerosols, sprays, powder for inhalation, extended-release preparations, and controlled-release preparations.

In the present disclosure, all components or reagents are commercially available products well known to those skilled in the art unless otherwise specified.

The technical solutions of the present disclosure will be clearly and completely described below with reference to the examples of the present disclosure. All other embodiments obtained by those skilled in the art based on the embodiments of the present disclosure without creative efforts shall fall within the protection scope of the present disclosure.

### Example 1

1. The mRNA and Protein Levels of p55γ Gene During Ferroptosis of Cardiomyocytes

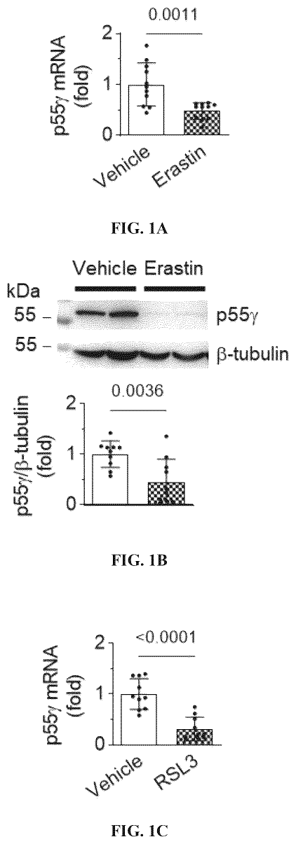

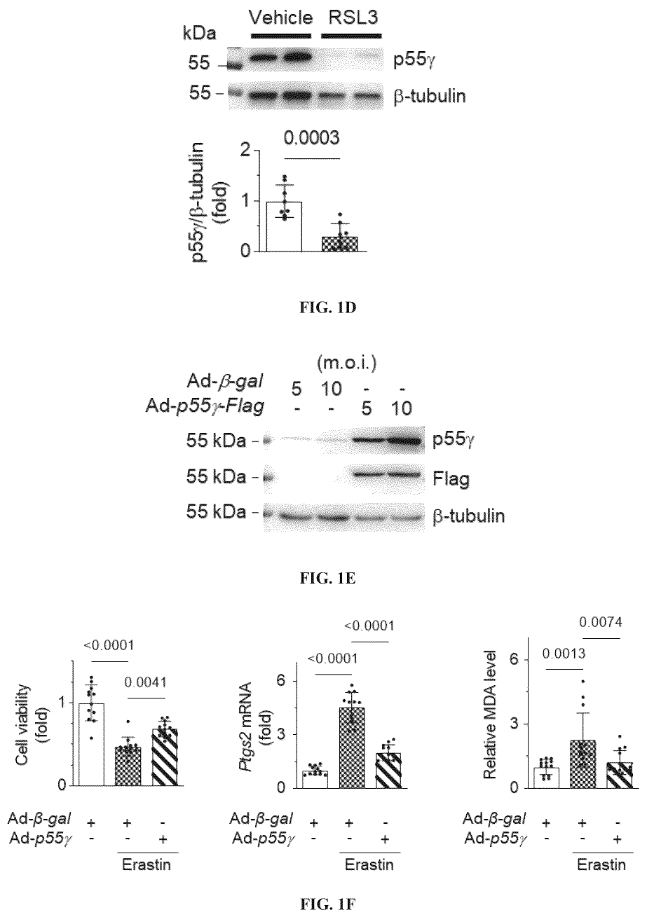

The cultured cardiomyocytes were divided into: an erastin group and a vehicle group, where the NRVMs were treated with erastin in the erastin group, while the cardiomyocytes in the vehicle group were not treated in any way. The mRNA and protein levels of p55γ gene in the two groups of cardiomyocytes were detected by conventional quantitative polymerase chain reaction (qPCR) and Western blot methods. It was found that the mRNA and protein levels of p55γ gene were significantly reduced in erastin-treated NRVMs (FIG. 1A and FIG. 1B).

The cultured cardiomyocytes were divided into: an RSL3 group and a vehicle group, where the RSL3 group treated NRVMs with another classic ferroptosis inducer (RSL3), and the vehicle group did not receive any treatment. The mRNA and protein levels of p55γ gene in the two groups of cardiomyocytes were detected by conventional qPCR and Western blot methods. It was found that the mRNA and protein levels of p55γ gene were also down-regulated in NRVMs treated with RSL3, another classic ferroptosis inducer (FIG. 1C and FIG. 1D). These data indicated that the occurrence of ferroptosis in cardiomyocytes was closely related to the downregulation of p55γ gene expression.

3. Influence of Overexpression of p55γ Gene on Ferroptosis of Cardiomyocytes

The p55γ adenovirus (Ad-p55γ-flag) was thawed on ice, the NRVMs were taken out, the original medium was removed by a suction pump, a serum-free DMEM medium was added, and the virus was diluted and added into a culture dish or well plate, mixed well, and placed in a CO₂ incubator for 48 h. Ad-p55γ-flag-transfected NRVMs were obtained. The level of p55γ protein in cardiomyocytes 48 h after infection with Ad-p55γ was detected by conventional Western blot method. Western blot analysis showed that p55γ protein levels were significantly increased (FIG. 1E). After overexpression of p55γ and β-gal in cardiomyocytes, erastin injury treatment was conducted, and a control group, β-gal experimental group, and p55γ overexpression group were set up. The viability of cardiomyocytes infected with Ad-p55γ after erastin injury was detected by cellular ATP content assay, the mRNA level of ptgs2 was detected by qPCR, and the lipid peroxidation of the cells was detected by intracellular MDA content assay. The results showed that compared with the control vector, overexpression of the p55γ gene could inhibit the erastin-induced decrease in cell viability, the increase in ptgs2 mRNA levels, and the increase in MDA content (FIG. 1F).

These data indicate that overexpression of p55γ may protect cardiomyocytes from erastin-induced cardiomyocyte ferroptosis.

### Example 2 The Role of p55γ Gene in Cardiac Ferroptosis Induced by I/R Injury

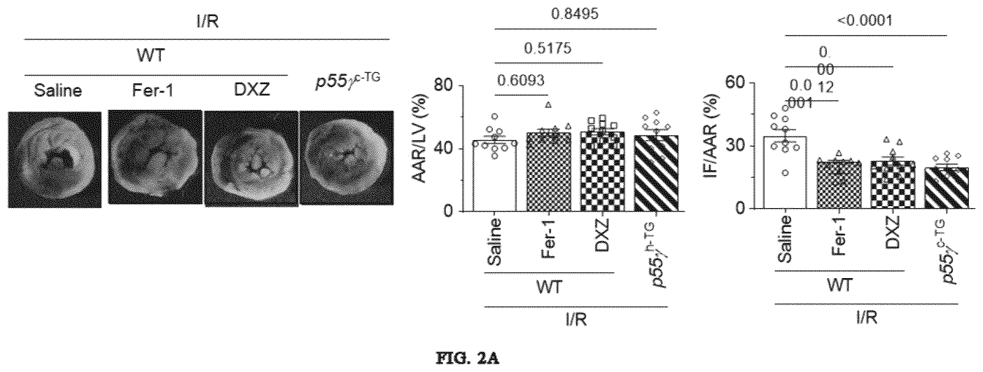

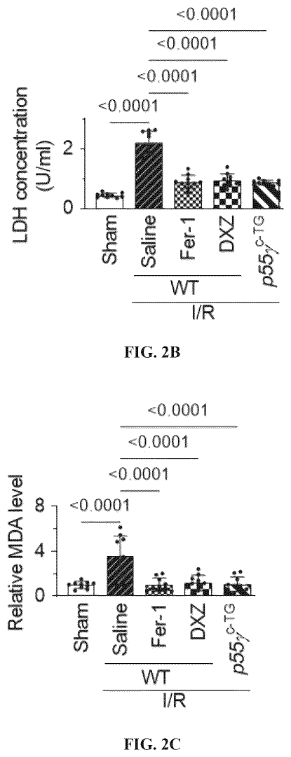

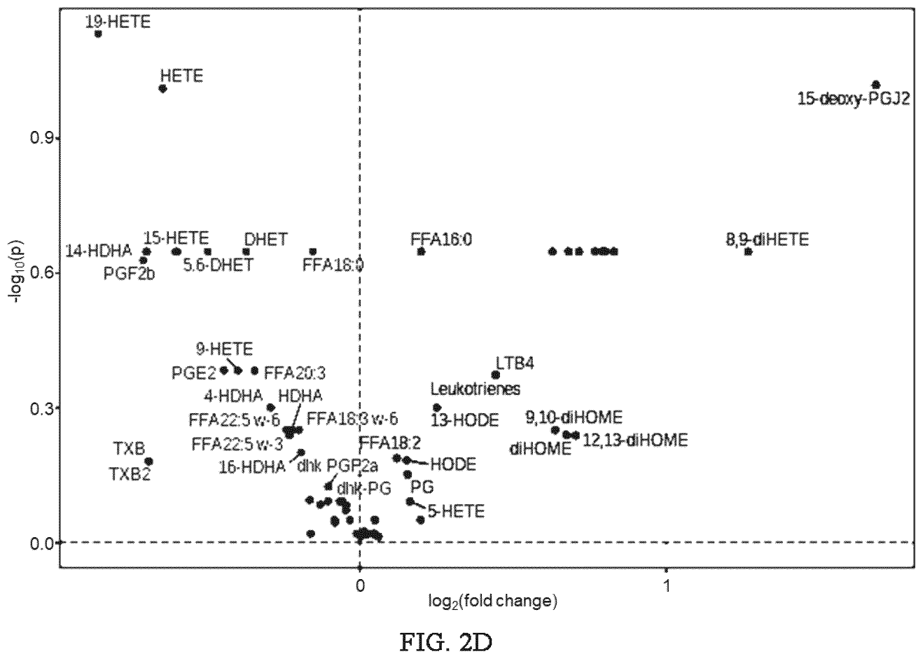

p55γ transgenic (p55γ^{h-TG}) mice were established according to routine methods in the art. Male wild-type (WT) C57 mice and p55γ^{h-TG} mice aged 8 to 12 weeks were selected to set up a sham operation control group and experimental groups, respectively, to construct I/R injury models. TTC staining, MDA detection, and oxidized lipid detection were conducted according to conventional methods in the art. To determine whether p55γ protein could inhibit I/R injury-induced cardiac ferroptosis, the experiments were carried out with the following groups: mice treated with saline, mice treated with Ferrostatin-1 (Fer-1) and Dexrazoxane (DXZ), and p55γ^{h-TG} mice. The cardiac infarction area of mice was detected by conventional TTC staining and the serum LDH concentration was assayed by LDH release kits. The results showed that the mice with p55γ^{h-TG} has a reduced I/R-induced myocardial infarct size and LDH concentration, and the therapeutic effect of which was comparable to that of the known ferroptosis inhibitors Fer-1 and DXZ treatment (FIGS. 2A and 2B). In addition, lipid peroxidation in the hearts of mice in each group was detected by intracellular MDA content assay. The results showed that as with Fer-1 and DXZ treatments, the MDA content (the most common by-product of lipid peroxidation) was significantly reduced in p55γ^{h-TG} mice compared with WT mice (FIG. 2C). Given that the peroxidation of polyunsaturated fatty acids (PUFAs) is an important step in ferroptosis, and in order to further clarify the role of p55γ gene in ferroptosis, oxidized lipid substances in the hearts of mice from the p55γ^{h-TG}-I/R group and WT-I/R group were measured using liquid chromatography-tandem mass spectrometry (LC-MS/MS). It was shown that the p55γ^{h-TG} group had reduced levels of 9-HETE, 12-HETE, 15-HETE, and 19-HETE (FIG. 2D).

Taken together, these data suggest that overexpression of p55γ protects heart from I/R-induced ferroptosis.

### Example 3 Study on the Potential Mechanism of p55γ Gene Against Ferroptosis

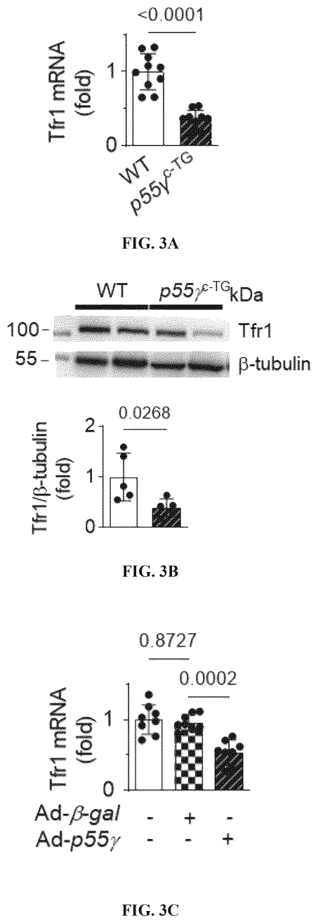

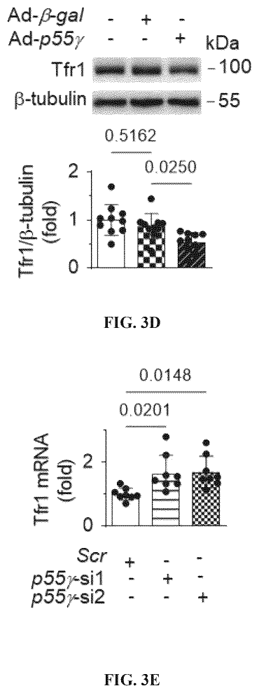

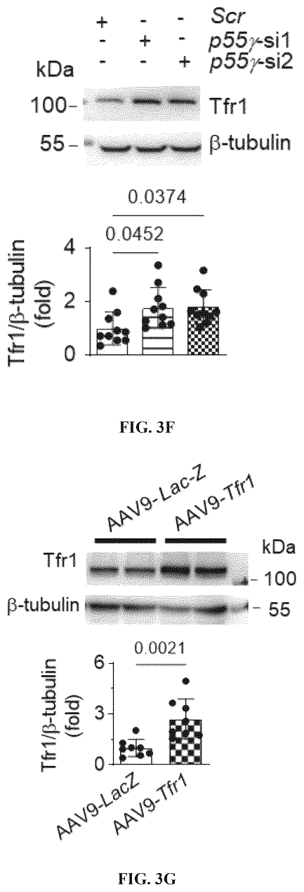

1. It was reported that transferrin receptor (TFRC, also known as transferrin receptor protein 1, Tfr1) was a specific ferroptosis marker. To investigate whether Tfr1 was involved in p55γ-induced cardioprotection, the heart tissues of WT and p55γ^{h-TG} mice were detected by qPCR and Western blot. It was shown that Tfr1 mRNA and protein levels were reduced in the hearts of p55γ^{h-TG} mice (FIG. 3A and FIG. 3B).

3. The mRNA and protein levels of Tfr1 in cardiomyocytes infected with Ad-p55γ for 48 h or p55γ siRNA for 72 h were detected by qPCR and Western blot. Two small interfering RNAs were designed for the coding region of the p55γ gene to specifically knock down the expression level of p55γ gene, called p55γ-si1 and p55γ-si2. The sequences were as follows: p55γ-si1 (5′-3′) sense strand GAAACAGUCCUUCAUUCCGTT (SEQ ID NO: 1); antisense strand CGGAAUGAAGGACUGUUUCGU (SEQ ID NO: 2); p55γ-si2 (5′-3′) sense strand GAGACAUUUCCAGGGAAGAGGUAAA (SEQ ID NO: 3); antisense strand UUUACCUCUUCCCUGGAAAUGUCUC (SEQ ID NO: 4). Double-stranded RNA-negative control of non-specific sequence (called Scrambled) was used as a control. RNAimax® was used to transfect NRVMs, and a control group and a p55γ knockdown group were set up. The results showed that overexpression of p55γ gene down-regulated Tfr1, while knockdown of p55γ gene up-regulated Tfr1 at the mRNA and protein levels (FIG. 3D to FIG. 3G). These results suggested that Tfr1 might play a key role in p55γ gene-mediated resistance to ferroptosis.

4. An adeno-associated virus 9 (AAV9) overexpression system was constructed to achieve heart-specific overexpression of Tfr1 in mice, and the heart 4 weeks after AAV9-Tfr1 infection was detected using Western blot. The results showed that the above treatment of mice achieved cardiac-specific overexpression of Tfr1 (FIG. 3G).

5. The WT mice and p55γ^{h-TG} mice infected or not infected with AAV9-Tfr1 were subjected to I/R injury treatment, and the cardiac infarct size and AAR of the above mice were calculated. It was shown that I/R injury was not reduced in p55γ^{h-TG} mice injected with AAV9-Tfr1 compared with that in p55γ^{h-TG} mice injected with a control virus (FIG. 3H). To determine whether Tfr1 mediated the effects of p55γ gene against ferroptosis, several parameters of ferroptosis were assessed, including ptgs2 mRNA levels, MDA production, and iron content in the hearts of WT mice and p55γ^{h-TG} mice infected or not infected with AAV9-Tfr1 for I/R injury (FIG. 3I). It was found that cardiac overexpression of Tfr1 blocked the anti-ferroptosis effect of p55γ gene.

These findings suggest that the anti-ferroptosis effect of p55γ may be attributed to the downregulation of Tfr1.

### Example 4 p55γ Gene Affecting the Expression of Tfr1 mRNA by Affecting Its Stability

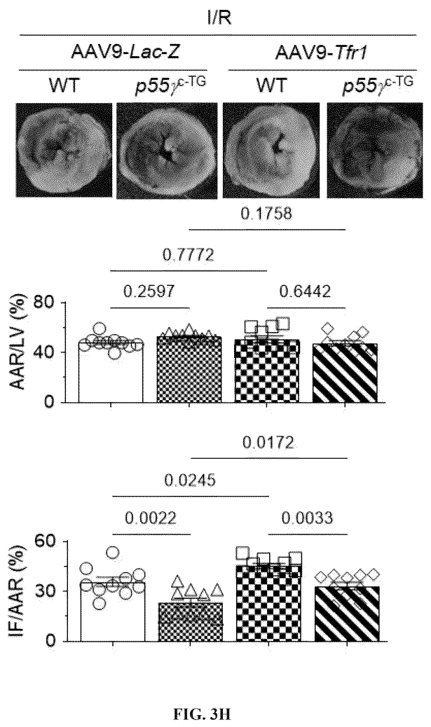

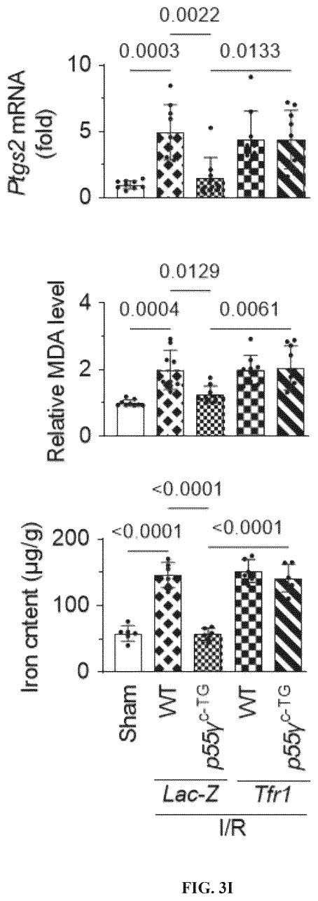

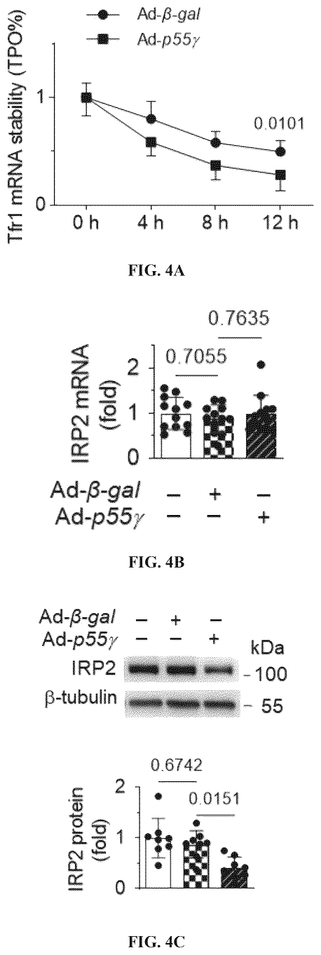

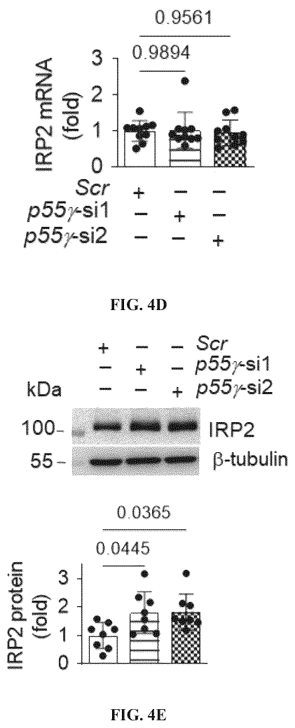

In order to further explore the specific molecular mechanism of p55γ gene down-regulating Tfr1, cardiomyocytes were transfected with p55γ adenovirus (Ad-p55γ-flag) and control virus (Ad-β-gal), and actinomycin D was added to inhibit the transcription in cells of each group, and then the stability of Tfr1 mRNA was compared in the two groups. The results showed that overexpression of p55γ gene led to a decrease in Tfr1 mRNA stability (FIG. 4A).

To elucidate whether the p55γ gene could regulate the mRNA stability of Tfr1through the IRPs-IRE system, the expression of IRP2 in NRVMs was evaluated. Ad-p55γ was used to infect cardiomyocytes, and qPCR and Western blot were conducted to detect Ad-p55γ-infected cardiomyocytes for 48 h. The results showed that p55γ gene overexpression resulted in a decrease in IRP2 protein levels, while its mRNA levels remained unchanged (FIG. 4B and FIG. 4C). In addition, the cardiomyocytes infected with p55γ siRNA for 72 h were detected by qPCR and Western blot, and it was shown that knockdown of p55γ gene led to an increase in IRP2 protein levels but did not change its mRNA levels (FIG. 4D and FIG. 4E). These results indicated that overexpression of the p55γ gene led to downregulation of IRP2 at the post-transcriptional level.

Ad-p55γ-infected cardiomyocytes were treated with MG132. Western blot was conducted to detect IRP2 protein levels, and it was shown that MG132 effectively eliminated the p55γ-mediated decrease in IRP2 protein (FIG. 4F). In order to further study whether p55γ could promote the ubiquitination and degradation of IRP2, the target gene sequences of p55γ, IRP2, and UB were found in NCBI. A pcDNA3.1(+) vector was used, sequence analysis software was used to find the restriction endonuclease cleavage sites present in the target gene sequences, and plasmids for p55γ, IRP2, and I/B were constructed. The plasmids were separately transfected into HEK293T cells, which were collected 24 h later and IRP2 ubiquitination was detected. Western blot was conducted to detect the ubiquitination level of IRP2. It was shown that p55γ gene overexpression increased the ubiquitination of IRP2 in HEK293T cells (FIG. 4G), suggesting that p55γ gene overexpression down-regulated IRP2 through the ubiquitin-proteasome pathway.

Western blots were conducted to detect IRP2 or Tfr1 protein levels in cardiomyocytes infected with Ad-p55γ and/or Ad-IRP2; meanwhile, the ptgs2 mRNA levels and cell viability were measured in cardiomyocytes infected with Ad-p55γ and/or Ad-IRP2 after H/R injury. It was shown that IRP2 gene overexpression counteracted both the downregulation of Tfr1 and the inhibition of H/R-induced ferroptosis in cardiomyocytes induced by p55γ gene overexpression (FIG. 4H to FIG. 4K).

Collectively, these data suggest that p55γ antagonizes the ferroptosis by promoting ubiquitination-mediated degradation of IRP2.

### Example 5 TRIM28 Mediates the Role of p55γ Gene Overexpression-Induced IRP2Degradation and Ferroptosis Resistance by Interacting with IRP2 and p55γ

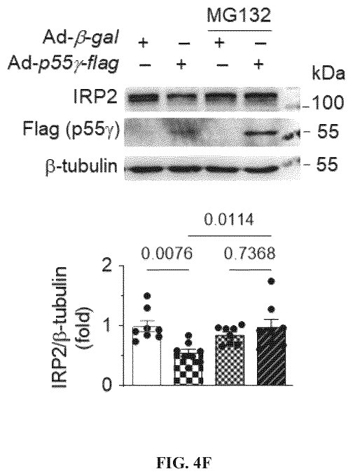

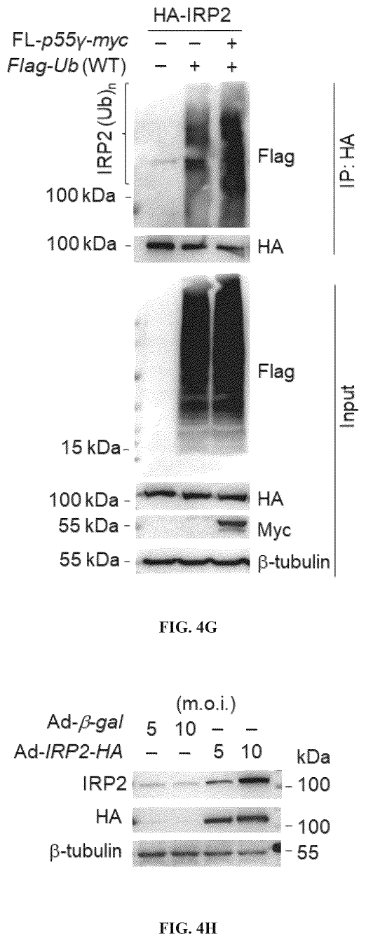

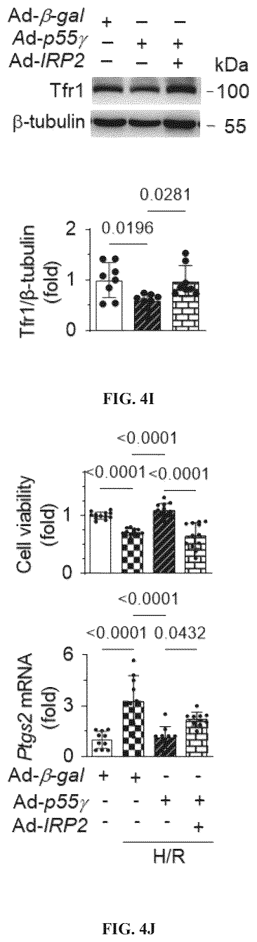

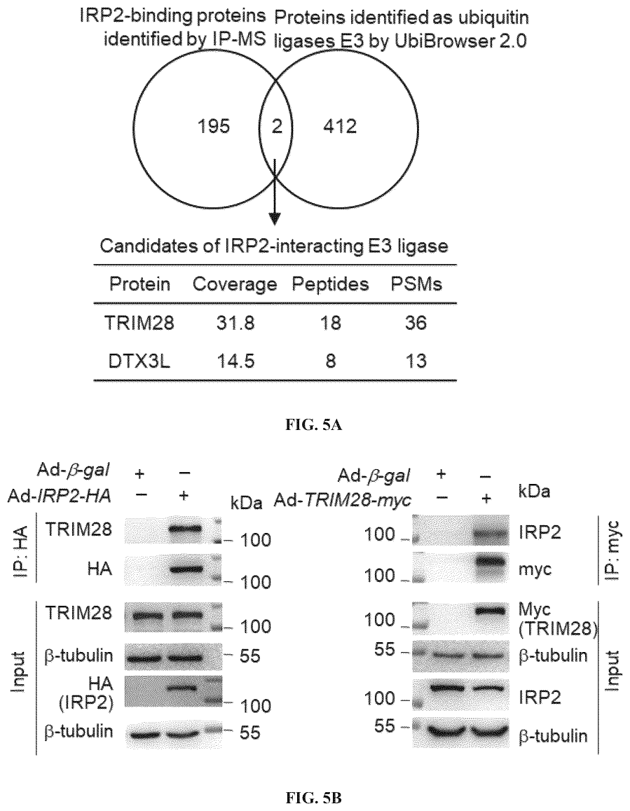

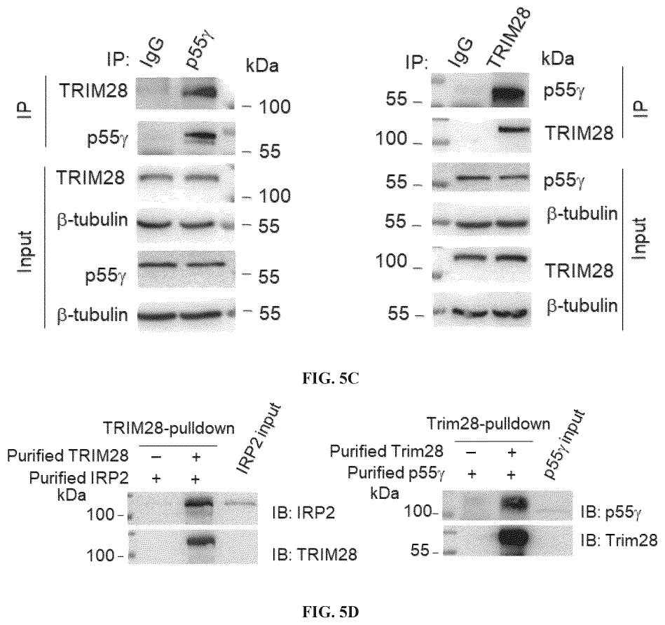

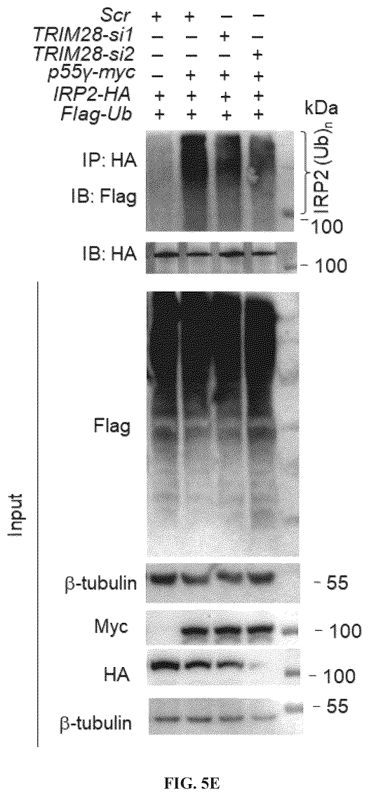

To further explore the mechanism of p55γ-mediated downregulation of IRP2, HA-tagged IRP2 was transfected into cardiomyocytes, and complexes containing IRP2 were purified with HA antibody for immunoprecipitation-mass spectrometry (IP-MS) analysis. The potential binding proteins obtained by the IP-MS were intersected with a protein identified as the ubiquitin ligase E3 by UbiBrowser 2.0. Analysis revealed TRIM28 as a potential E3 ligase of IRP2 (FIG. 5A).

In order to study the role of TRIM28 on IRP2 ubiquitination and degradation and how TRIM28 regulates IRP2, co-immunoprecipitation experiments were conducted using HA-tagged IRP2. Experimental results showed that IRP2 and TRIM28 interacted to form a complex (FIG. 5B). In addition, an interaction between the endogenous p55γ and TRIM28 was also observed through the co-immunoprecipitation experiments (FIG. 5C). Further confirmation of direct interaction of TRIM28 with IRP2 and p55γ was obtained using the purified recombinant protein (FIG. 5D). Next, our study sought to determine the involvement of TRIM28 in degradation of IRP2 and consequential cardioprotective effects mediated by p55γ. As expected, knocking down TRIM28 led to the attenuation of the p55γ overexpression-induced augmentation of IRP2 ubiquitination in HEK293T cells (FIG. 5E) and diminished the p55γ overexpression-induced decrease in IRP2 and Tfr1 protein level (FIG. 5F).

These results indicate that the TRIM28 is a necessary molecule for p55γ overexpression-induced IRP2 degradation and ferroptosis resistance.

### Example 6

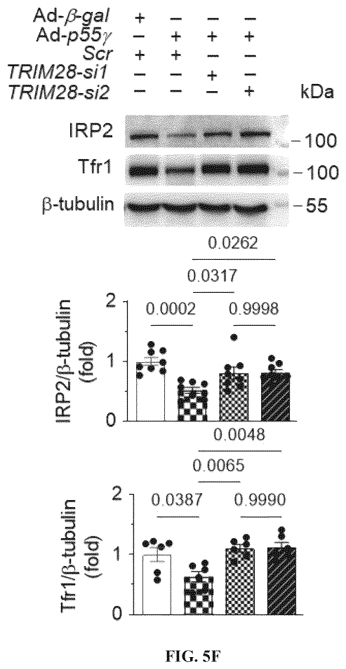

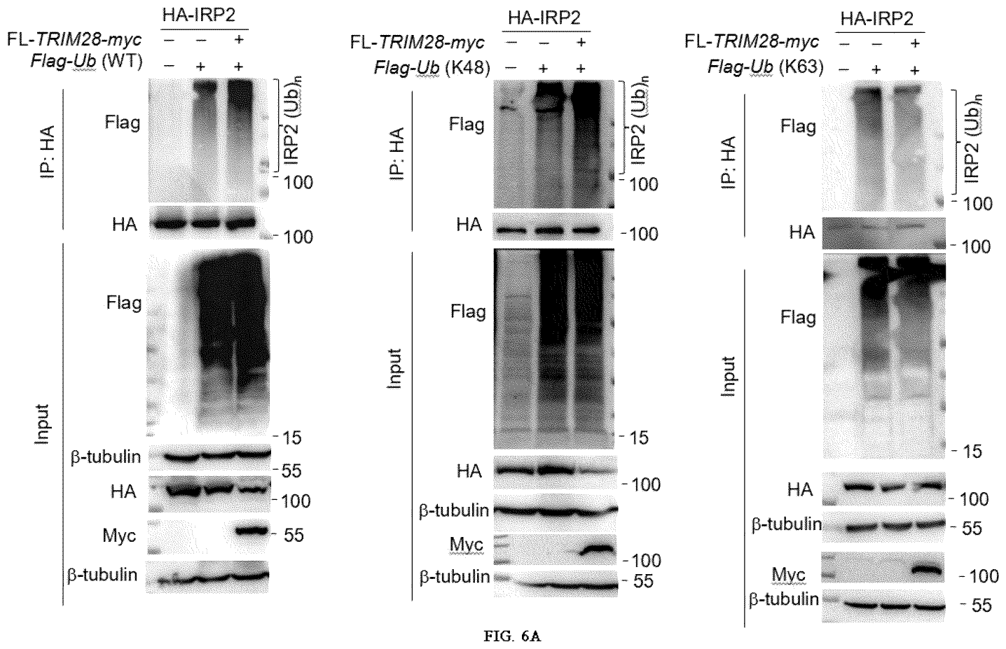

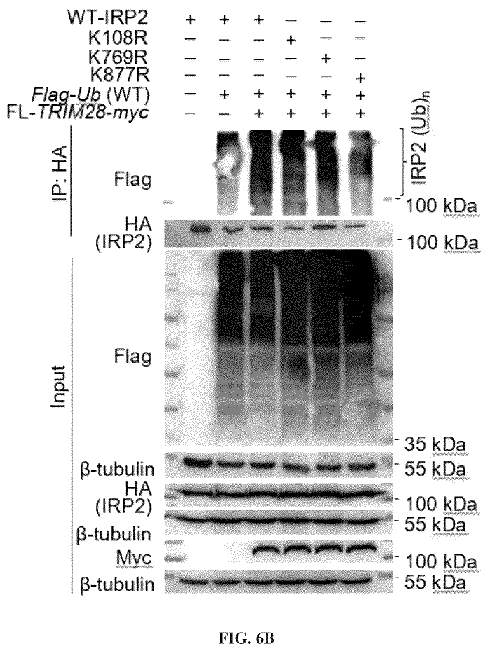

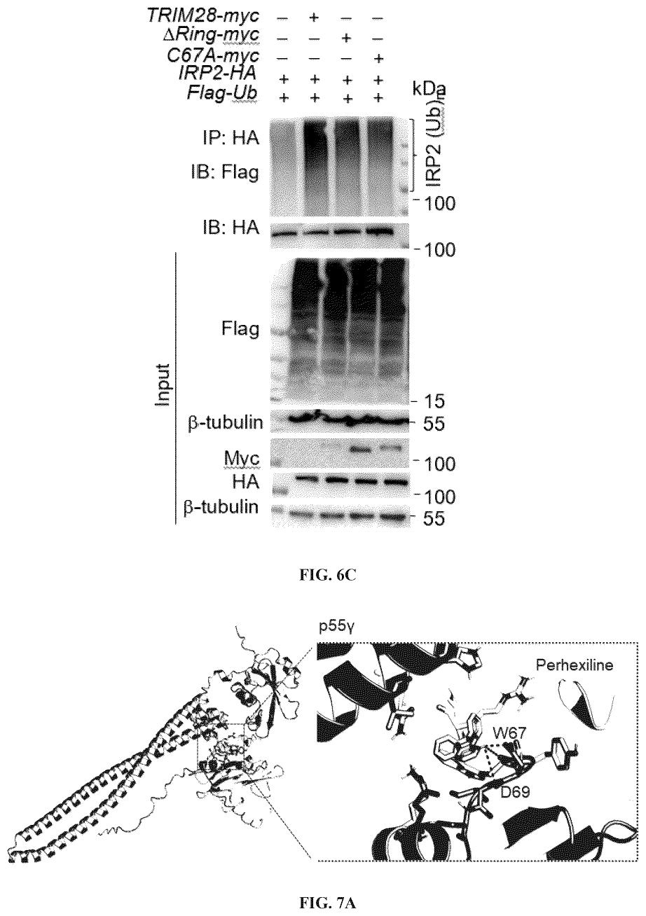

In order to further reveal the ubiquitin modification of TRIM28 on IRP2, HEK293 cells were co-transfected with myc-tagged TRIM28, HA-tagged IRP2, and Flag-tagged ubiquitin plasmids. It was found that TRIM28 significantly increased the ubiquitination of IRP2 in the presence of WT ubiquitin and K48-linked ubiquitin chains, without affecting the K63-linked ubiquitination of IRP2 (FIG. 6A), indicating that TRIM28 specifically promotes K48-linked ubiquitination of IRP2, rather than K63-linked ubiquitination.

Proteomic analysis of quantitative ubiquitination modification showed ubiquitination of three lysine residues K108, K769, and K877 of IRP2. To determine which lysine site of IRP2 was responsible for TRIM28 mediated ubiquitination, mutant plasmids of IRP2 (K108R, K769R, and K877R) were generated by site-directed mutagenesis and the lysine residues at specific sites were replaced with arginine residues. In HEK293 cells, after co-transfection of the IRP2 mutant with MYC-tagged TRIM28 and Flag-tagged ubiquitin, it was observed that TRIM28 lost its ubiquitination function in the K877R mutant, indicating that K877 is the specific site for TRIM28 to modify IRP2 ubiquitination (FIG. 6B). These findings indicate that TRIM28 may promote K48 polyubiquitination at the K877 site of IRP2.

In order to further determine the specific amino acid site of TRIM28 responsible for IRP2 ubiquitination, a full-length TRIM28 (FL-TRIM28) plasmid was constructed, as well as two mutant plasmids: one lacking RING domain (ΔRing), and the other with a cysteine to arginine mutation at position 67 (C67A). The results showed that both the ΔRing and C67A mutants failed to induce IRP2 ubiquitination (FIG. 6C). Together, these data suggest that TRIM28 may inhibit I/R-induced myocardial ferroptosis by promoting ubiquitin degradation of IRP2 in a RING domain-dependent manner.

### Example 7

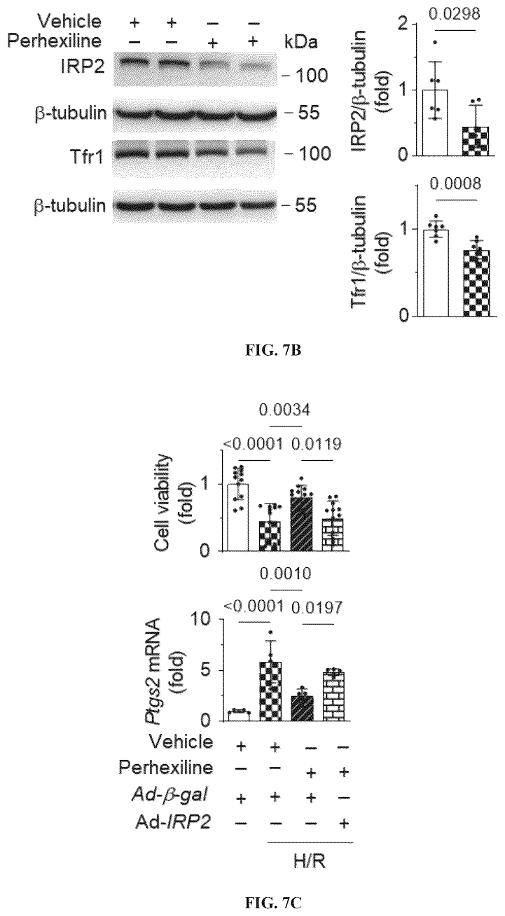

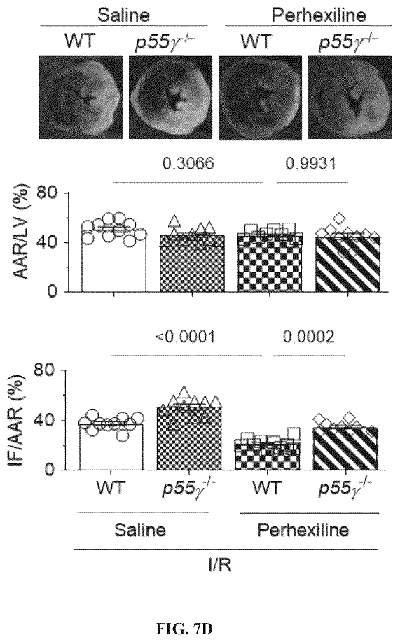

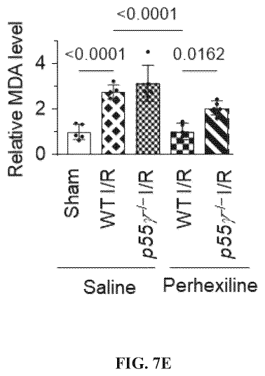

In view of the importance of p55γ in combating ferroptosis, it is particularly important to find potential activators of p55γ for the treatment of I/R injury. Therefore, potential activators of p55γ were predicted using existing small molecule drug libraries. Subsequently, molecular docking analysis was conducted based on the crystal structure of p55γ. The docking analysis revealed that perhexiline exhibited significant binding affinity to p55γ, forming intermolecular hydrogen bonds with the Trp67 and Asp69 amino acid residues in p55γ (FIG. 7A). To determine the potential role of perhexiline in cardiomyocyte ferroptosis, experiments were first done in cardiomyocytes. Western blot experiment results showed that the protein levels of Tfr1 and IRP2 were significantly down-regulated after perhexiline treatment in cardiomyocytes for 24 h (FIG. 7B). The effect of perhexiline was further tested in the context of H/R injury model. The results showed that perhexiline treatment significantly blocked the decrease in cell activity and the increase in ptgs2 mRNA levels induced by H/R injury. Overexpression of IRP2 could block the inhibitory effect of perhexiline on H/R induced decrease in cell activity and increase in ptgs2 mRNA levels (FIG. 7C).

p55γ knockout (p55γ^{-/-}) mice were established according to routine methods in the art. Male wild-type (WT) C57 mice and p55γ^{-/-} mice aged 8 to 12 weeks were selected to set up a sham operation control group and experimental groups, respectively, and the mice were intraperitoneally injected with perhexiline 24 h and 2 h before constructing the I/R injury model. TTC staining and MDA detection were conducted according to conventional methods in the art. The results of TTC staining and MDA detection showed that perhexiline pretreatment could significantly reduce the myocardial infarct size in mice and reduce the level of MDA in the heart of mice (FIG. 7D and FIG. 7E), whereas this cardioprotective effect was abolished in p55γ^{-/-} mice (FIG. 7D and FIG. 7E).

In summary, the present disclosure elucidates p55γ as a novel inhibitory factor of cardiomyocyte ferroptosis. Overexpression of p55γ leads to Tfr1 downregulation through the IRP2 post-transcriptional system. The present disclosure further proposes TRIM28 as a novel E3 ligase of IRP2, where p55γ enhances ubiquitination and subsequent degradation of IRP2 by binding to TRIM28, thereby contributing to the downregulation of Tfr1 expression and ultimately inhibiting ferroptosis.

Various cardiovascular diseases are accompanied by imbalances in iron homeostasis. During myocardial injury, excessive iron ions accumulate in the heart, causing disruption of iron homeostasis and abnormal expression of iron homeostasis-related proteins. I/R injury is also one of the most common forms of myocardial injury. The p55γ protein is an important regulator of cardiac iron homeostasis and can resist cardiac ferroptosis induced by I/R injury. Thus, the present disclosure highlights the potential of p55γ as a key regulator of I/R injury-induced cardiomyocyte death and demonstrates the potential application of targeting p55γ in maintaining cardiac iron homeostasis and treating related diseases.

The above descriptions are merely preferred embodiments of the present disclosure. It should be noted that a person of ordinary skill in the art may further make several improvements and modifications without departing from the principle of the present disclosure, but such improvements and modifications should be deemed as falling within the protection scope of the present disclosure.

1. A method for maintaining cardiac iron homeostasis, comprising using p55γ gene and/or p55γ protein as a target.

2. The method according to claim 1, wherein promotion of activity of the p55γ protein or promotion of expression of the p55γ gene maintains the cardiac iron homeostasis and treats a related disease.

3. The method according to claim 1, wherein transfection of an adenovirus overexpressing the p55γ gene maintains the cardiac iron homeostasis and treats a related disease.

4. The method according to claim 1, wherein overexpression of the p55γ gene leads to downregulation of transferrin receptor 1 (Tfr1) through an iron-regulatory protein 2 (IRP2) post-transcriptional system, thereby maintaining the cardiac iron homeostasis and treating a related disease.

5. The method according to claim 1, wherein the p55γ protein increases ubiquitination and subsequent degradation of IRP2 by binding to tripartite motif containing 28 (TRIM28) protein, thereby downregulating expression of Tfr1 and ultimately inhibiting ferroptosis to maintain the cardiac iron homeostasis and treat the related disease.

6. The method according to claim 1, wherein the p55γ protein inhibits cardiomyocyte ferroptosis.

7. A method for screening a drug for maintaining cardiac iron homeostasis and treating a related disease, comprising
predicting a potential activator of p55γ protein using small molecule drug libraries;
conducting molecular docking analysis based on the crystal structure of p55γ protein;
selecting an activator that binds to the p55γ protein and forms intermolecular hydrogen bonds with the Trp67 and Asp69 amino acid residues in the p55γ protein;
detecting if the activator down-regulates expressions of Tfr1 and IRP2 in cardiomyocytes;
detecting if the activator decreases cell activity and increases ptgs2 expression in mice with hypoxia and reoxygenation (H/R) injury;
detecting if the activator reduces a myocardial infarct size in mice and reduces a level of MDA in the heart of p55γ gene knockout (p55γ^{-/-}) mice with ischemia and reperfusion (I/R) injury; and
determining the activator as the drug when the activator shows positive results in all detections.

8. The method according to claim 7, wherein the drug promotes expression of the p55γ gene and/or promotes activity of the p55γ protein to maintain the cardiac iron homeostasis and treat the related disease.

9. The method according to claim 7, wherein the drug comprises perhexiline.

10. A method for treating cardiac iron homeostasis-related disease, comprising administering a therapeutically amount of a drug that promotes activity of p55γ protein or promotes expression of p55γ gene.

11. The method according to claim 10, wherein the drug comprises an adenovirus overexpressing the p55γ gene.

12. The method according to claim 11, wherein overexpression of the p55γ gene leads to downregulation of transferrin receptor 1 (Tfr1) through an iron-regulatory protein 2 (IRP2) post-transcriptional system, thereby maintaining the cardiac iron homeostasis and treating a related disease.

13. The method according to claim 10, wherein the cardiac iron homeostasis-related disease is ferroptosis-related disease.

14. The method according to claim 10, wherein transfection of an adenovirus overexpressing the p55γ gene maintains the cardiac iron homeostasis and treats a related disease.

15. The method according to claim 10, wherein overexpression of the p55γ gene leads to downregulation of transferrin receptor 1 (Tfr1) through an iron-regulatory protein 2 (IRP2) post-transcriptional system, thereby maintaining the cardiac iron homeostasis and treating a related disease.

16. The method according to claim 10, wherein the p55γ protein increases ubiquitination and subsequent degradation of IRP2 by binding to tripartite motif containing 28 (TRIM28) protein, thereby downregulating expression of Tfr1 and ultimately inhibiting ferroptosis to maintain the cardiac iron homeostasis and treat the related disease.

17. The method according to claim 10, wherein the p55γ protein inhibits cardiomyocyte ferroptosis.

18. The method according to claim 10, wherein the drug comprises perhexiline.
